(19) United States
(12) Patent Application Publication
(10) Pub. No.: US 2025/0157664 A1
(43) Pub. Date: May 15, 2025

(54) Quantification of Lung Fibrosis

(71) Applicant: QUREIGHT LIMITED, Cambridge (GB)

(72) Inventors: Alessandro Ruggiero, Cambridge (GB); Fahdi Kanavati, Cambridge (GB); Muhunthan Thillai, Cambridge (GB)

(21) Appl. No.: 18/937,659

(22) Filed: Nov. 5, 2024

(30) Foreign Application Priority Data
Nov. 9, 2023 (GB) 2317160.6

(51) Int. Cl. G16H 50/30; G06N 3/0464; G06T 7/00; G16H 30/40

(52) U.S. Cl. CPC G16H 50/30; G06N 3/0464; G06T 7/0012; G16H 30/40

(57) ABSTRACT

A system and method for computing a lung fibrosis metric are described. The system has an input interface to receive initial lung imaging data for the patient, a trained neural network lung segmentation model to generate lung segmentation data from the initial lung imaging data, a fibrosis model pre-processor to apply the lung segmentation data to the lung imaging data to produce modified lung imaging data, a trained neural network lung fibrosis model to generate fibrosis segmentation data from the modified lung imaging data, a fibrosis model post-processor to process the fibrosis segmentation data in combination with the lung segmentation data to generate labelled voxel data, a fibrosis metric processor to use the labelled voxel data to compute a fibrosis volume metric for the patient, and an output interface to provide the fibrosis volume metric as the lung fibrosis metric for the patient.

### TECHNICAL FIELD

The present application relates to a system, apparatus, and computer-implemented method for computing a lung fibrosis metric. Examples described herein apply a set of trained models to compute a lung fibrosis metric based on initial lung imaging data.

### BACKGROUND

Lung diseases are some of the most common medical conditions affecting millions of people around the world. Typical lung disease may be caused by internal and external factors such as smoking, infections, drug-induced side effects, and gene mutations.

Interstitial lung disease (ILD) is a category of lung disease that affects the interstitium, a thin, delicate lining between alveoli of the lung, whereby tiny blood vessels traverse and permit gas transfer between the alveoli and blood.

One type of ILD is Idiopathic Pulmonary Fibrosis (IPF), with its cause unknown. The NIH's National Library Medicine, states there are ˜100,000 IPF patients, (˜30,000-40,000 new cases/year). The global IPF treatment market is currently $3.2bn and it is expected to increase to $4.3bn by 2026), predominantly in American and European regions (Expert Market Research, Global IPF Treatment Market Outlook). Market drivers include increasing incidence of complex respiratory diseases, ageing, and awareness. In 2018, 79 drug and computational technology products were being developed for IPF. On Feb. 3, 2021, 2,613 industrially funded clinical trials were in setup, recruitment or active trial status for Complex respiratory diseases and 109 studies for IPF.

Patients with IPF show progressive scarring of tissue that could lead to respiratory failure and even death if untreated. There has been no proven treatment for IPF (or other fibrotic lung diseases) until 2014, with the advent of two landmark drugs, Roche's Pirfenidone and Nintedanib from Boehringer Ingelheim. Pirfenidone has shown to reduce the decline in lung capacity/function caused by IPF. In addition to showing similar efficacy as Pirfenidone, Nintedanib demonstrates effectiveness towards non-IPF forms of ILD. Since 2014, over 50 drug candidates have undergone (or presently undergoing) clinical trials for IPF worldwide.

Measuring Forced Vital Capacity (FVC) via a breathing test is a standard approach for diagnosing IPF in hospitals. FVC is a measure of full lung capacity and thus correlates with lung decline. As the fibrosis worsens in the lung, the lung begins to shrink, and FVC decreases. A reduction in the rate of decline of FVC is indicative of a certain level of efficacy from a candidate drug. In clinical trials, FVC is normally used as a primary end-point.

To obtain an accurate measure of FVC is difficult, however. It is equally difficult to assess the progression of the disease solely based on measuring FVC. Thus, there are concerns with FVC as a clinical end-point. These concerns include 1) the breath test is variable depending on the technician who performs it, 2) the test varies within the patient depending on how well or unwell the patient is on the day, and 3) it may not be a true and accurate representation of fibrosis with respect to computed tomography (CT) scans.

WO2023/281252 A2 describes a machine learning approach for preparing a model for assessing the progression of a lung disease. This patent publication describes the training of an “airway model” and a “lung model” that may be used to track the progression of lung disease. The airway model is trained to detect and label the airways of the lungs from CT scan data. Changes in the airways may then be used to track the progression of a disease. The lung model is trained to detect and label lung lobes from the same CT scan data. The labelled portions of the lung may then be used in trial, testing, or screening. The patent publication describes how a “human-in-the-loop” approach may be to check and correct errors or inconsistencies in initial model segmentations. The methods of the patent publication provide accurate models for airway segmentation and lung segmentation.

EP 4,246,434 A1 describes a computer-implemented method for quantifying and predicting the progression of interstitial lung disease. The computer-implemented method applies a convolutional neural network (CNN) that takes a two-dimensional axial slice of a scan and outputs a segmentation for the slice. The segmentation is in the form of a multi-channel probability map of abnormality regions. This multi-channel probability map is transformed into lung and reticulo-vascular masks by thresholding. A weighting may be applied to identified reticulo-vascular structures and used to quantify an extent of fibrotic disease and/or predict the progression of an ILD.

The paper “Lung Infection Quantification of COVID-19 in CT Images with Deep Learning” by Fei Shan et al., published on ArXiv on 10 Mar. 2020, describes the use of a “VB-Net” deep learning architecture to classify COVID-19 infection regions. The VB-Net architecture receives CT data and predicts areas of infection.

The paper “Relational Modeling for Robust and Efficient Pulmonary Lobe Segmentation in CT Scans” by Weiyi Xie et al., published on ArXiv on 12 May 2020, describes a pulmonary lobe segmentation model that may be applied to CT data. The pulmonary lobe segmentation model uses a relational two-stage U-Net configuration to label lobes of the lung from CT data. The two-stages relate to global and local features. The labelled lobes may then be used to help with treatment of COVID-19.

Given the above, there is a need for apparatus that can quantify lung fibrosis in a simple and straightforward manner. Equally, there is an unmet need for a more efficient way to quantitatively assess lung disease progression in response to the dosage of a new drug candidate in the context of clinical trials.

It is understood that the examples described below are not limited to implementations, which solve any, or all of the disadvantages of the exemplary known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides a machine learning approach to assess the presence and progression of fibrosis. The presence and progression of fibrosis may be assessed for a patient based on lung imaging data, such as computed tomography (CT) scans. The present disclosure describes a black-box system that may be applied within research or clinical settings to allow researchers and medical professionals to quantify lung fibrosis. In examples set out herein, lung fibrosis is quantified using a lung fibrosis metric. The lung fibrosis metric is computed based on a fibrosis volume metric representing a proportion of lung volume that exhibits fibrosis. The fibrosis volume metric is computed based on two machine learning models: a first machine learning model that is trained to predict lung segmentation data (e.g., that identifies portions of lung imaging data as relating to the lung) and a second machine learning model that is trained to predict fibrosis segmentation data (e.g., that identifies portions of lung imaging data as exhibiting fibrosis).

In a first aspect of the described examples, a system for computing a lung fibrosis metric for a patient comprises an input interface, a trained neural network lung segmentation model, a fibrosis model pre-processor, a trained neural network lung fibrosis model, a fibrosis model post-processor, a fibrosis metric processor, and an output interface. The input interface is configured to receive initial lung imaging data for the patient. The trained neural network lung segmentation model is configured to generate lung segmentation data from the initial lung imaging data. The lung segmentation data indicates which portions of the lung imaging data relate to lung features of the patient. The fibrosis model pre-processor is configured to apply the lung segmentation data to the lung imaging data to produce modified lung imaging data. The trained neural network lung fibrosis model is configured to generate fibrosis segmentation data from the modified lung imaging data. The fibrosis segmentation data indicates which portions of the modified lung imaging data relate to fibrosis features. The fibrosis model post-processor is configured to process the fibrosis segmentation data in combination with the lung segmentation data to generate labelled voxel data. The fibrosis metric processor is configured to use the labelled voxel data to compute a fibrosis volume metric for the patient. The fibrosis volume metric represents a proportion of lung volume that exhibits fibrosis. The output interface is configured to provide the fibrosis volume metric as the lung fibrosis metric for the patient.

The first aspect, as described herein, provides a straightforward, interpretable metric relating to fibrosis that may be used to identify and track disease progression. The lung fibrosis metric allows large-scale trials to reliably compare different patients, e.g. to determine the efficacy of a new treatment in a drug trial. The lung fibrosis metric is stable and consistent over different sets of patients. This is achieved by the system architecture presented herein. The use of a trained neural network lung segmentation model allows lung segmentation data to be used as a form of attention map that improves the training and inference of the trained neural network lung fibrosis model. The two models thus interact synergistically. By identifying portions of lung imaging data as “lung”, the lung segmentation data may be used to inform the lung fibrosis model of areas of the lung imaging data that relate to the lung (and this may be performed probabilistically in certain examples). This provides improved fibrosis segmentation data (e.g., more accurate and repeatable identification of fibrosis) that is then also used in combination with the original lung segmentation data to compute a volume metric. The volume metric may be independent of lung size and so comparable across different patients. Further, by providing a black-box system, a lung fibrosis metric may be computed while limiting the need for expensive and time-consuming radiologist or expert guidance. This then enables rapid and cheap diagnosis.

In a second aspect of the described examples, a computer-implemented method of computing a lung fibrosis metric for a patient is provided. The method comprises receiving initial lung imaging data for the patient; applying a trained neural network lung segmentation model to the initial lung imaging data to generate lung segmentation data, the lung segmentation data indicating which portions of the lung imaging data relate to lung features of the patient; applying the lung segmentation data to the lung imaging data to produce modified lung imaging data; applying a trained neural network lung fibrosis model to the modified lung imaging data to generate fibrosis segmentation data, the fibrosis segmentation data indicating which portions of the modified lung imaging data relate to fibrosis features; processing the fibrosis segmentation data in combination with the lung segmentation data to generate labelled voxel data; computing a fibrosis volume metric for the patient using the labelled voxel data the fibrosis volume metric representing a proportion of lung volume that exhibits fibrosis; and providing the fibrosis volume metric as the lung fibrosis metric for the patient.

The second aspect provides advantages that are similar to the first aspect.

In a third aspect of the described examples, a computer-implemented method of configuring a lung fibrosis metric system is provided. The method comprises: obtaining lung segmentation training data comprising lung imaging data with lung segmentation annotations; training a neural network lung segmentation model based on the lung segmentation training data to produce a set of trained lung segmentation model parameters, said training comprising optimising a loss function, the loss function being computed based on a comparison of a ground-truth lung segmentation annotation and a predicted lung segmentation annotation from the neural network lung segmentation model; obtaining fibrosis training data comprising lung imaging data with fibrosis segmentation annotations; and training a neural network lung fibrosis model based on the fibrosis training data, said training comprising optimising a loss function, the loss function being computed based on a comparison of a ground-truth fibrosis segmentation annotation and a predicted fibrosis annotation from the neural network lung fibrosis model. The predicted fibrosis annotation is computed by: computing lung segmentation data using the neural network lung segmentation model instantiated with the trained lung segmentation model parameters as applied to a sample from the lung imaging data; pre-processing the sample from the lung imaging data using the lung segmentation data to generate a modified sample; and applying the neural network lung fibrosis model in a training mode to the modified sample to generate the predicted fibrosis annotation.

The third aspect provides a two-stage training approach that allows an accurate lung fibrosis detection system to be easily trained. The training approach uses two sets of annotated data—annotated lung segmentation data and annotated fibrosis segmentation data. These sets of data may be independent and based on different available training sets. The training approach then uses this annotated data to train two neural network models in a manner that improves the accuracy and reliability of at least the lung fibrosis neural network model.

In certain examples, a neural network lung fibrosis model may be trained using a training set of lung imaging data that is annotated by medical experts. The medical experts may be one or more expert thoracic radiologists. The training data may include sets of CT images of lungs from different patients affected by fibrotic lung disease. The annotation may comprise the expert thoracic radiologists indicating areas of fibrosis within the lung imaging data. This may involve marking areas on a CT image. A lung segmentation neural network model may be trained using the methods described in WO2023/281252 A2 and/or based on a training set of lung imaging data that is annotated to indicate areas of the lung or different identified areas of the lung.

In certain examples, training may involve “human-in-the-loop” refinement. For example, a digital dataset that comprises manual annotations performed by a group of expert thoracic radiologists may be first used to initially train the fibrosis model. This model may be then applied to a set of unsegmented images to generate an estimated set of segmented images. The resulting segmentation may then be corrected by a group of expert thoracic radiologists and the correction encoded as part of a loss function. The fibrosis model may then be retrained and updated, where this process may be iteratively performed multiple times.

Discussion of “fibrosis” in the present disclosure relates to tissue of the lung that is deemed by a medical expert to be “fibrotic”. Fibrosis tissue may comprise a spectrum of radiological appearances ranging from mild inflammatory changes (e.g., that appear as so-called “ground glass changes”), to mild fibrotic changes (e.g., that are associated with reticulation) to more severe fibrotic changes (e.g., those that are referred to as “honeycombing”).

Certain examples described herein use a multi-stage system where each stage relies on multiple convolutional neural networks arranged in a pipeline. Different parts of the pipeline may be arranged to process lung imaging data at different resolutions. Preferably, neural network models used herein operate on three-dimensional data, wherein lung imaging data is represented as a three-dimensional volume that reflects a scanned CT volume, said volume resulting from a scan of one or more lung or lung regions.

The methods described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor (such as one or more graphical processing units) or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software, which “describes” or defines the configuration of hardware, such as HDL (hardware description language) software, as is used for designing silicon chips, or for configuring universal programmable chips, to carry out desired functions. The models described herein may be implemented as a combination or hardware and software (e.g., a processor implementing computer program code) and a set of configuration data that comprises values for a set of trainable parameters for the models.

The preferred features described in the following section may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1 is a schematic diagram illustrating an example of a system for computing a lung fibrosis metric;

FIGS. 2A and 2B are pictorial diagrams illustrating an example of unsegmented and segmented images of lungs on axial and coronal planes, respectively;

FIG. 3A is a schematic diagram illustrating an example of a trained neural network lung fibrosis model;

FIG. 3B is a schematic diagram illustrating an example of a processing block within the trained neural network lung fibrosis model;

FIG. 4 is a flow diagram illustrating an example computer-implemented method of computing a lung fibrosis metric for a patient;

FIG. 5 is a flow diagram illustrating an example computer-implemented method of configuring a lung fibrosis metric system; and

FIG. 6 is a block diagram of a computing device suitable for implementing embodiments of the invention.

Common reference numerals are used throughout the figures to indicate similar features.

### DETAILED DESCRIPTION

Examples of the present invention are described below. These examples represent the suitable modes of putting the invention into practice that are currently known to the applicant, although they are not the only ways in which this could be achieved. The description sets forth the functions of the example and the sequence of steps for constructing and operating the example. However, the same or equivalent functions and sequences may be accomplished by different examples.

In comparative and past approaches, lung disease progression in patients in routine clinical practice is typically assessed visually by expert thoracic radiologists. For each patient, the experts may study up to 300 two-dimensional axial images produced from their computed tomography (CT) scan before making visual assessments based on the appearance of areas within the images while noting any observable changes over time. Fibrosis progression may take the form of an architectural distortion of the lung tissue, where normal lung tissue is replaced with fibrosis (i.e., fibrotic tissue) and where lung volume loss may occur at the late stages of disease. This progression may be discerned through visual comparison of different marked up two-dimensional images taken at different times. The manual quantitative analysis of the radiological findings is possible and this would involve the use of a graphical user interface to digitally identify the areas of interest within the three-dimensional CT scan.

These comparative and past approaches present problems. For example, visual assessment can only yield qualitative data. Analysis of images from a CT scan by eye has also been shown to be subjective. It is also susceptible to the physical state of the radiologist at the time of analysis. There can thus be considerable variation between radiologists in their interpretation of the same dataset (or even between annotations of the same radiologist at different points in time). Different experience levels can also make a difference to the annotation of fibrosis and progression determinations. Thus, with limited expert resources, accurate assessment/analysis takes a long time. Pools of consultant radiologists with a range of experience levels may be able to reduce variation, but such pools are expensive (in both person-hours and cost) and suffer from social factors (such as group dynamics and deference to authority). This can make it difficult and long-winded to test new treatments over large cohorts (e.g., hundreds or thousands of patients).

For this reason, quantitative analysis of fibrosis is highly desirable for measurements of the fibrotic tissue to become integrated into assessments regarding patient disease progression and drug response.

Computer assisted diagnosis (CAD) algorithms for classifying fibrosis do exist. However, there is currently no consensus regarding an optimal method for use in clinical trials and clinical practice. Existing commercial algorithms have been developed with old computational approaches. One of the earliest methods was based simply on density measurements on CT scans—these are not precise measurements as density is not specific to fibrosis and also normal or other pathological structures may have density similar to fibrosis. Other methods included Adaptive Multiple Features Method (AMFM), a computer-based analysis tool that quantifies lung patterns using image patches extracted from images and CALIPER (Computer-Aided Lung Informatics for Pathology Evaluation and Rating), a computer-vision based technique that includes volumetric local histogram and morphologic analysis to provide quantitative assessment of pulmonary parenchymal disease on CT data. The Quantitative Lung Fibrosis (QLF) tool was introduced 10 years ago and it is a technique based on Support Vector Machine (SVM) which is a process that calculate texture features and run a SVM classifier—this was developed in Scleroderma lung disease and not IPF. Newer approaches are related to Data-driven Textural Analysis (DTA) is based on unsupervised feature learning and is implemented as a simple convolutional neural network (CNN). All these approaches rely on measurements of individual voxels therefore results can be affected by noise and different reconstruction algorithms.

Another approach uses Functional Respiratory imaging, consisting in acquiring inspiratory and expiratory datasets and calculating volumes and calibre of the airway which are less dependent on noise and reconstruction algorithms but measure the by-product of fibrosis (airway dilatation) rather the fibrosis itself. For example, the “airway model” described in WO2023/281252 A2 may be used to measure airway dilation.

Finally, there are certain open-source algorithms, the workings and training of which are similar to existing commercial algorithms. They lack consistency and standardisation when used to assess a specific disease. Also, open-source approaches lack the critical mass of training set data generated by multiple vendors and different acquisition settings. Also they lack expert input to achieve accurate, reliable performance and indeed makes such open-source algorithms unsuitable for use with images in the real world.

The present invention described herein overcomes at least some of the shortcomings present in the existing approaches/algorithms by providing an improved way to identify and assess the extension of fibrosis in the lungs in a quantitative manner using machine learning. One or more machine learning models are hereby trained to quantify the analysis of lung imaging data such that the resultant quantification produced, e.g. in the form of a lung fibrosis metric, may be used as or adapted to be a biomarker for assessing fibrotic lung disease (i.e., IPF) progression in place of or in addition to the standard Forced Vital Capacity (FVC) in clinical trials or routine clinical practice. The present invention provides the following advantages over existing approaches.

These advantages include, but are not limited to:
a) The lung fibrosis metric may be a more sensitive marker than FVC for early IPF progression (useful to monitor ‘mild’ patients who are starting to progress);
b) The lung fibrosis metric is a closer representation of the CT radiology change in fibrosis than FVC;
c) The lung fibrosis metric may supplement FVC as a more sensitive marker of progression;
d) The lung fibrosis metric may be used to provide a prediction of mortality by quantifying the extent of fibrosis; and
e) The lung fibrosis metric may allow cohort selection, i.e. identify patients on trials who are progressing their fibrosis so are best suited for treatment

In certain example, the machine learning models comprise neural network architectures. The neural network architectures may comprise an arrangement of processing units, where at least one of the processing units comprises parameters for a computation (such as a matrix multiplication or other transformation) and where the parameters may be optimised with a training procedure based on a loss function.

The machine learning models may be trained iteratively using segmented data generated by expert thoracic radiologists. The combination of the models is configured to provide a rapid, consistent, automatic, accurate segmentation and a measurement of fibrosis for images from lung disease patients. This then allows confident assessments to be made regarding the extent of patient lung fibrosis and progression and treatment response based on fibrosis volume changes. The lung fibrosis metric thus serves as a sensitive biomarker in the clinical study and treatment of fibrotic lung disease.

For a machine learning model for assessing extent and distribution of fibrosis, herein referred to as a fibrosis model, the training data may comprise a set of CT images. These CT images are identified and labeled based on the progression of the fibrotic lung disease. The CT images are cross-sectional images of an entire or selected body part generated by a computed tomography scanner. The training set suitably captures the appearances of the lungs and expert radiologists can read scans and identify semi-quantitatively the distribution and severity of the abnormalities. The fibrosis on these CT images in a training set may be segmented by a team of expert radiologists. On this, a neural network architecture suitable for image segmentation is trained.

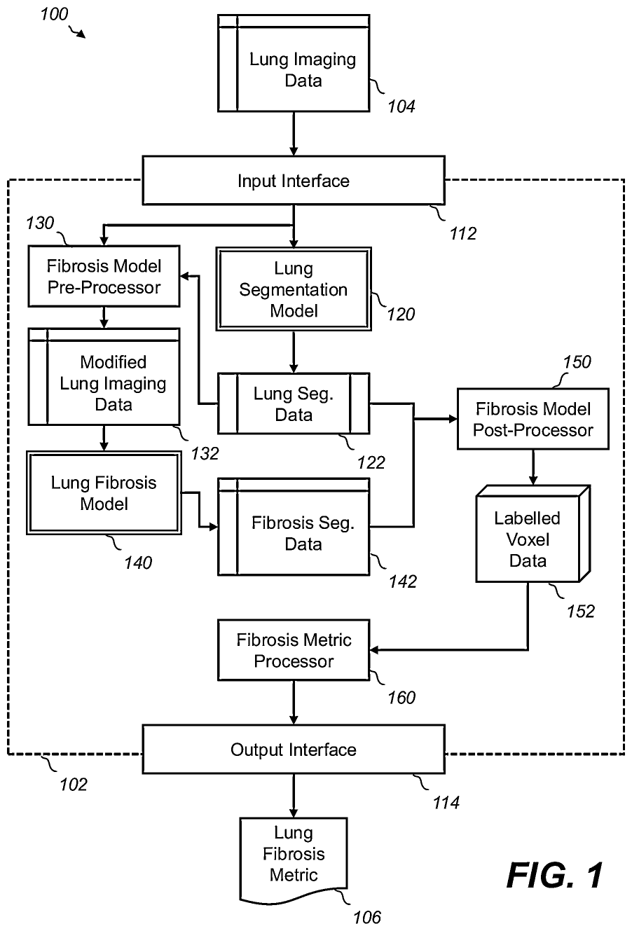

FIG. 1 shows an example 100 of a system 102 for computing a lung fibrosis metric for a patient. The system 102 operates on lung imaging data 104, which is processed to produce a lung fibrosis metric 106.

The lung imaging data 104 may comprise data measured from the patient, such as scan data. The scan data may comprise CT imaging data. The scan data may be provided as one or more two-dimensional images (e.g., matrices of single or multi-channel data) and/or as a three-dimensional data structure (e.g., representing a set of slices through the lung or lungs). In preferred examples, the lung imaging data 104 comprises a three-dimensional matrix (sometimes called a “tensor” in machine learning), with one or more channels of data representing a scan intensity or value. For example, the lung imaging data 104 may be provided as a three-dimensional matrix of values (i.e., a set of voxels) having a defined (voxel) spacing. Often CT imaging data is provided as greyscale values and so may be provided as a single channel. The resolution of lung imaging data (e.g., number of elements in each of the spatial dimensions) may depend on the source of the data (e.g., the resolution of the imaging equipment and/or the post-processing imaging software). Lung imaging data 104 may be provided within a defined three-dimensional extent or envelope. For example, CT scan data may be provided as data values within a cylinder representing a volume of the scan.

The lung fibrosis metric 106 may comprise a single scalar value that may be used to compare an extent of fibrosis between different patients. In a base case, the lung fibrosis metric 106 may provide a single value for a set of lungs of the patient. In an extended case, the lung fibrosis metric 106 may comprise a plurality of metric values for different portions of a lung or set of lungs. The lung fibrosis metric 106 is based on a fibrosis volume metric that represents a proportion of lung volume that exhibits fibrosis. The lung fibrosis metric 106 may be comparable to a fibrosis volume metric that is calculated based on a set of manual radiologist fibrosis annotations. This is possible because the system 102 comprises neural network models that are trained on at least radiologist (i.e., expert) fibrosis annotations. In this manner, the lung fibrosis metric 106 may be used in studies that compare results to experiments where manual fibrosis segmentation has been performed. The lung fibrosis metric 106 is further interpretable as an easy-to-understand metric for the diagnosis of disease and to track disease progression. Disease progression is further facilitated by the reliability of the outputs of the system 102.

Returning to FIG. 1, the system 102 comprises an input interface 112 and an output interface 114. The input interface 112 receives the lung imaging data 104. The lung imaging data 104 may be referred to as “initial” lung imaging data 104 as it reflects a set of lung imaging data as obtained prior to processing within the system 102. The output interface 114 returns or outputs the lung fibrosis metric 106 from the system 102. The term “interface” is used herein to refer to any physical and/or logical interface that allows for one or more of data input and data output. An interface may be implemented by retrieving data from one or more memory locations, as implemented by a processor executing a set of instructions. An interface may also comprise physical couplings over which data is received. An interface may comprise an application programming interface and/or method call or return. For example, in a software implementation an interface may comprise passing data and/or memory references to a function initiated via a method call; in a hardware implementation, an interface may comprise a wired interconnect between different chips, chipsets or portions of chips.

Following receipt, the lung imaging data 104 is passed to a lung segmentation model 120 and a fibrosis model pre-processor 130. Preferably, the lung segmentation model 120 and a fibrosis model pre-processor 130 process the lung imaging data 104 in parallel, but serial processing may also be used. The lung segmentation model 120 is a trained neural network lung segmentation model. The lung segmentation model 120 generates lung segmentation data 122 from the lung imaging data 104. Lung segmentation data 122 may comprise two or three-dimensional data with one or more channels that comprise values that indicate whether a data element belongs to a lung. Preferably the lung segmentation data 122 is the same resolution as the lung imaging data 104, but in certain examples the lung segmentation data 122 may comprise a different resolution (e.g., based on explicit or implicit up or down sampling within the lung segmentation model 120). The lung segmentation data 122 may comprise binary data values indicating whether a data element (e.g., a pixel or voxel) relates to a portion of a lung (e.g., “0” is “not lung” and “1” is “lung”). In preferred cases, the lung segmentation data 122 may comprise a probability map comprising data values (e.g., values between 0 and 1) representing a probability that a data element relates to a portion of a lung. In this case, a binary mask may be obtained by thresholding and/or sampling the probability values.

In certain variations, the lung segmentation data 122 may comprise values that indicate different areas or volumes of the lung. For example, the lung segmentation data 122 may comprise a set of channels where each channel relates to a lobe of the lung. In one case, there may be five channels, each channel representing a probability map for each of the five lung lobes (upper, middle, and lower lobes of the right lung and the upper and lower lobes of the left lung). In another case, there may be three channels representing upper, middle, and lower lobes and a single channel representing left and right lungs. In yet other cases, the lung segmentation data 122 may have further channels indicating lung segments (each lobe having two to five segments) and/or lobules. If the lung segmentation data 122 indicates classifications of particular lung portions (as described here), a single binary or probability value indicating a more general “lung” segmentation may be generated as an individual channel or as a function of a set of channels representing the particular lung portions (e.g., as a maximum value of the channels or the like). It may be understood that the precise form of the lung segmentation data 122 may depend on the implementation and may vary.

Turning back to FIG. 1, the fibrosis model pre-processor 130 is configured to pre-process the lung imaging data 104 to generate modified lung imaging data 132 prior to application of a lung fibrosis model 140. The lung fibrosis model 140 is also a trained neural network model. The lung fibrosis model 140 may or may not comprise the same neural network architecture as the lung segmentation model 120. In both cases, the lung fibrosis model 140 comprises a set of trained parameters that differs from the trained parameters of the lung segmentation model 120. It has been found that a common (i.e., using the same or overlapping components) neural network architecture may be used and then trained using different sets of training data (e.g., lung segmentation annotations and lung fibrosis annotations respectively). A common neural network architecture may simplify implementation, deployment, and engineering optimisation. The fibrosis model pre-processor 130 is configured to apply the lung segmentation data 122 to the lung imaging data 104 to produce modified lung imaging data 132. This may comprise multiplying the lung segmentation data 122 and the lung imaging data 104 (e.g., using a matrix multiplication). If the lung segmentation data 122 comprises a binary mask the fibrosis model pre-processor 130 may act to mask the lung imaging data 104 to leave values within the data that relate to portions of the lungs. In the case where, preferably, the lung segmentation data 122 comprises a probability map, the lung segmentation data 122 may act to weight the lung imaging data 104 to emphasis portions of the lung in the lung imaging data 104. In this case, the fibrosis model pre-processor 130 may act as a form of attention mechanism that uses the lung segmentation data 122 to attend to areas of the lung when applying the lung fibrosis model 140. In one case, said multiplication may be applied element-by-element, e.g. where a value of a data element in a three-dimensional matrix representing the lung imaging data 104 is multiplied by a value of a corresponding data element in a three-dimensional matrix representing the lung segmentation data 122. The corresponding data elements in this case may comprise corresponding voxels or corresponding pixels at a shared resolution.

In the system 102, the lung fibrosis model 140 is configured to generate fibrosis segmentation data 142 from the modified lung imaging data 132. The fibrosis segmentation data 142 indicates which portions of the modified lung imaging data 132 relate to fibrosis features. For example, the fibrosis segmentation data 142 may comprise a two or three-dimensional matrix with at least one data channel representing a fibrosis label. As for the lung segmentation data 122, the fibrosis segmentation data 142 may comprise binary data values indicating a classification of “fibrosis” or “not fibrosis” for a particular data element, where each data element relates to a potential portion of a lung of the patient, or may comprise a probability map (e.g., with values between 0 and 1) with values that represent a likelihood of a fibrosis classification. In one case, the fibrosis segmentation data 142 may be of the same, or a corresponding, resolution in two or three dimensions to the lung imaging data 104. Due to the pre-processing of the lung imaging data 104 with the lung segmentation data 122, there is no requirement to manually restrict application of the lung fibrosis model 140 to particular “lung” pixels or voxels—the data values for the fibrosis segmentation data 142 may be populated across the complete range of data elements for the initial lung imaging data 104 regardless of whether they show a portion of lung or not. This is because the application of the lung segmentation data 122 and the modified lung imaging data 132 guides the lung fibrosis model 140 (or, more precisely. guides the parameter values of the model during training) to effectively combine lung segmentation and fibrosis labelling functions. In this respect, data elements of the fibrosis segmentation data 142 that do not correspond to portions of the lung will be assigned low or zero values for fibrosis. In the case where the lung segmentation data 122 comprises a probability map, then the lung fibrosis model 140 may learn a complex non-linear function to determine fibrosis. For example, the lung fibrosis model 140 may be able to accommodate uncertainty with respect to lung segmentation (e.g., variety within lung segmentation values) and integrate both local and global patterns within two and/or three-dimensions. This all provides an accurate and reliable labelling of fibrosis within the fibrosis segmentation data 142.

Following the generation of the fibrosis segmentation data 142, this, and the lung segmentation data 122 are passed to a fibrosis model post-processor 150 to generate labelled voxel data 152. In a case where the lung segmentation model 120 and the lung fibrosis model 140 operate on a two-dimensional input (e.g., on two-dimensional CT images) then the components 120 to 140 may be applied iteratively (e.g., to a plurality of said images) to provide the lung segmentation data 122 and the fibrosis segmentation data 142 as a plurality of two-dimensional data “slices” for different planes through the volume of the lungs. In a preferred case, where the lung segmentation model 120 and the lung fibrosis model 140 operate on a three-dimensional input (e.g., a three-dimensional matrix) then the lung segmentation data 122 and the fibrosis segmentation data 142 may already comprise volumetric data. In cases where the lung segmentation data 122 and the fibrosis segmentation data 142 are of differing resolutions the fibrosis model post-processor 150 may perform two and/or three dimensional up or down sampling to provide the data within a common (i.e., shared resolution). In a preferred implementation, the lung segmentation model 120 and the lung fibrosis model 140 are configured to operate on three-dimensional data of a common (i.e., shared resolution), where that resolution may be set as a configuration parameter and/or set based on the resolution of the lung imaging data 104. In a case where the lung imaging data 104, the lung segmentation data 122, and the fibrosis segmentation data 142 have the same number of data elements in three-dimensions (i.e., the same three-dimensional resolution), this may facilitate computation and comparison. In one case, the fibrosis model post-processor 150 is configured to multiply corresponding data values for voxels in the lung segmentation data 122 and the fibrosis segmentation data 142. This may comprise a form of post-processing attention. This may act to refine the fibrosis segmentation data 142 based on portions of the data that are deemed to be “lung” in the lung segmentation data 122. In another case where the lung segmentation data 122 is a binary mask, the fibrosis model post-processor 150 may zero any values in the fibrosis segmentation data 142 that do not relate to the lung.

The labelled voxel data 152 comprises a set of data elements with a fibrosis value for each data element. For example, each voxel has a corresponding fibrosis value. The fibrosis model post-processor 150 may apply a thresholding procedure on continuous (e.g. float data type) fibrosis values (e.g., representing an output of a function applied of the lung segmentation data 122 and the fibrosis segmentation data 142) to output a binary “fibrosis” label for each data element. A binary “fibrosis” label may be equivalent to a radiologist deeming a portion of a CT scan as corresponding to fibrotic tissue. The labelled voxel data 152 may additionally comprise a label indicating portions of the lung, e.g. as based on the lung segmentation data.

In the example 100 of FIG. 1, the labelled voxel data 152 is received by a fibrosis metric processor 160. The fibrosis metric processor 160 is configured to compute a fibrosis volume metric from the labelled voxel data 152. The fibrosis volume metric represents a proportion of lung volume that exhibits fibrosis. In one case, the fibrosis volume metric may comprise a ratio of the number of voxels labelled as exhibiting fibrosis to the number of voxels labelled as relating to the lung. In one case, the labelled voxel data 152 may only comprise “lung” voxels. In this case, the fibrosis volume metric may comprise the number of voxels labelled as exhibiting fibrosis within the total number of voxels. The fibrosis volume metric may comprise a dimensionless percentage or ratio value (e.g., between 0 and 1 or 0 and 100%). Given this, the fibrosis volume metric may be compared across different patients and/or across time. In FIG. 1, the fibrosis metric processor 160 passes the fibrosis volume metric to the output interface 114 for output as the lung fibrosis metric 106.

In one variation where the lung segmentation model 120 is configured to classify data elements as relating to one or more anatomic portions of a lung (e.g., lobes, segments, and/or lobules), the fibrosis metric processor 160 may be configured to compute fibrosis volume metrics for those different anatomic portions. For example, the fibrosis model post-processor 150 may use the lung segmentation data 122 to set a multi-class label within the labelled voxel data 152 (e.g., as a set of multi-channel one-hot values and/or integer labels mapped by a dictionary to the different anatomic portions). The fibrosis metric processor 160 may then output fibrosis volume metrics for one or more labelled anatomic portions of the lungs. For example, fibrosis metric processor 160 may be configured to output fibrosis volume metrics for each lung lobe. This may be used to help diagnose different conditions and/or track the effectiveness of treatment in different areas of the lungs.

As described above, the lung imaging data 104 may comprise one or more of a set of computed tomography (CT) images and three-dimensional CT image data. For example, three-dimensional CT image data may be supplied in a NIfTI file format (e.g., in the form of “*.nii” files originally developed by the Neuroimaging Informatics Technology Initiative for neuroscience data) or a DICOM file format (named after a Digital Imaging and Communications in Medicine standard). Lung imaging data 104 may be provided in a compressed form and decompressed before use. In certain examples pre-processing may be applied when loading lung imaging data 104 to provide it as an accessible tensor in memory.

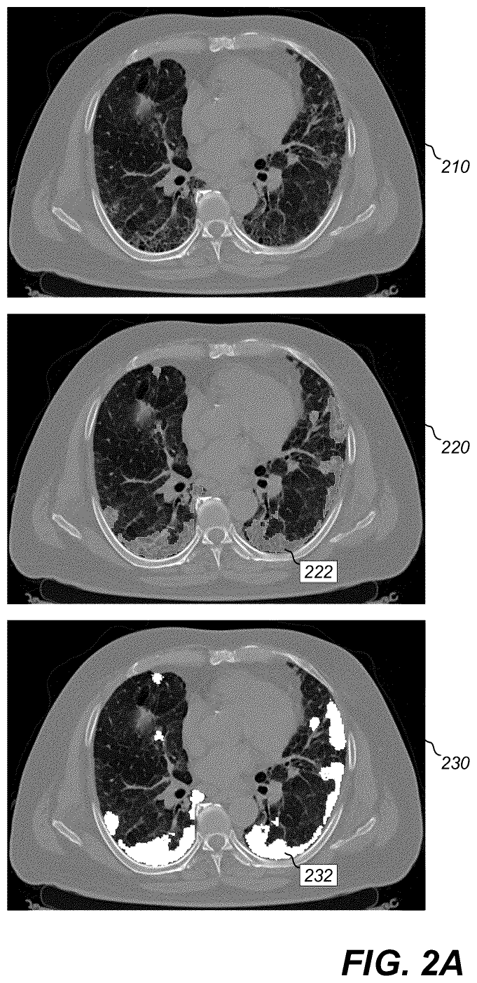

FIGS. 2A and 2B show examples of fibrosis segmentation data as predicted with a lung fibrosis model (such as lung fibrosis model 140 in FIG. 1). FIGS. 2A and 2B show two-dimensional CT images of a patient. These CT images may be standalone images or may comprise “slices” from three-dimensional CT imaging data. FIG. 2A shows various versions 210, 220, 230 of an axial slice for a 64-year old male patient with suspected lung fibrosis. FIG. 2B shows various versions 260, 270, 280 of a coronal slice for the same patient. The upper images 210 and 260 show unlabelled slices. For example, these slices may form part of the initial lung imaging data 104 in FIG. 1. The lower two images 220, 230 and 270, 280 then show these slices with additional fibrosis segmentation labelling. The second images, 220 and 270, show the fibrosis segmentation labelling as a semi-opaque overlay 222, 272. The third images, 230 and 280, show the same fibrosis segmentation labelling as an opaque white overlay 232, 282 (for better visibility in black and white). In this case, the fibrosis segmentation labelling may form part of the fibrosis segmentation data 142 in FIG. 1, or be based on the output labelled voxel data 152 as generated by the fibrosis model post-processor 150. In FIGS. 2A and 2B, the fibrosis segmentation is binary (i.e., “fibrosis” or “not fibrosis”) and is applied to individual data elements (which may correspond to pixels or pixel areas in the lung imaging data). Two-dimensional slices of fibrosis segmentation labelling may be generated by taking slices through output labelled voxel data 152 along the same planes as used for the axial and coronal slices. By comparing the upper and lower images in FIGS. 2A and 2B, it may be seen how the fibrosis segmentation labelling as generated by the methods and systems described herein identifies areas of fibrosis that are visible in the raw initial images (typically as honeycomb and reticulation areas).

FIGS. 2A and 2B show how fibrosis often manifests differently in different portions of the lungs. In FIG. 2A, the areas of fibrosis are seen at the rear of the lungs with different manifestations in left and right lungs. In FIG. 2B, there are again different manifestations in left and right lungs, but also more prominent fibrosis in the lower portions of the lung. Hence, variations that output a fibrosis volume metric for particular lung portions (e.g., for each lung lobe, segment, and/or lobule) may help a non-radiologist (e.g., a General Practitioner, technician, or nurse) easily visualise the extent of fibrosis or its progression over time. These local fibrosis volume metrics may be provided together with a single global fibrosis volume metric. Any of these metrics may be repeated computed over time to evaluate progression of IPF.

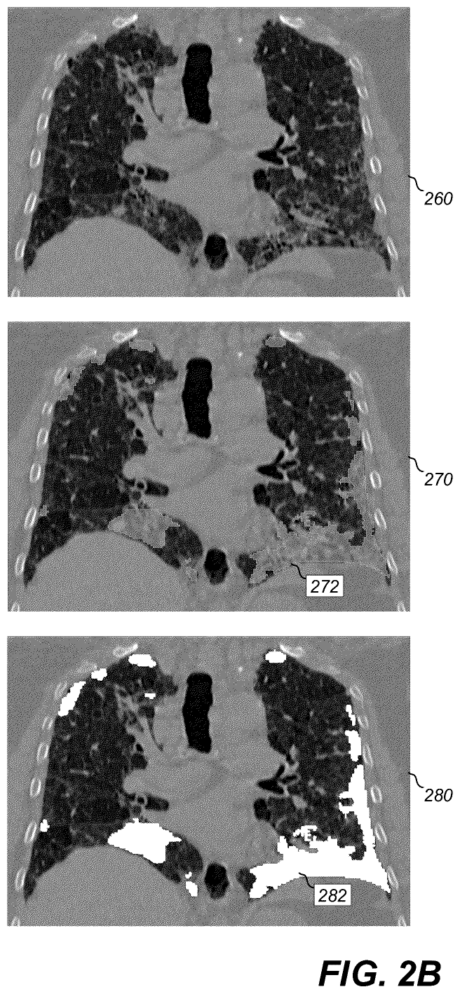

FIGS. 3A and 3B show a specific example 300 of a neural network architecture that may be used to implement at least the lung fibrosis model 140 of FIG. 1 (or used within the later methods). In certain cases, the same architecture may be used to implement the lung segmentation model 120 (but with different sets of trained parameters). Example dimensions and feature sizes are shown to help explain the operation of the architecture but these should not be taken as limiting. Those skilled in the art will appreciate that dimensions and tensor sizes may be configured based on particular implementations and preferences.

The terms “neural network model” and “neural network architecture” are used interchangeably herein to refer to a set of one or more artificial neural networks that are configured to perform a particular data processing task, in this case tasks to label fibrosis and/or lung portions. A “neural network architecture” may comprise a particular arrangement of one or more neural network layers of one or more neural network types. Neural network types include convolutional neural networks, recurrent neural networks, and feed-forward neural networks. The present examples are based on convolutional neural networks. Convolutional neural networks involve the application of one or more convolution operations. Convolutional neural network layers may be applied on two-dimensional or three-dimensional data values. Convolutional neural network layers may have stride parameters and a filter size. Neural network architectures that are described herein may be implemented using known programming tools and libraries including PyTorch® and TensorFlow®.

A “neural network layer”, as typically defined within machine learning programming tools and libraries, may be considered an operation that maps input data to output data. In many machine learning programming tools and libraries, neural network layers of different types are predefined within the library, and may be configured as part of a neural network architecture via a model definition. The model definition may, for example, be in the form of a Python class. A “neural network layer” may apply one or more weights to map input data to output data. One or more bias terms may also be applied. The weights and biases of a neural network layer may be applied using one or more multidimensional arrays or matrices. In general, a neural network layer has a plurality of parameters whose values influence how input data is mapped to output data by the layer. These parameters may be trained in a supervised manner by optimizing an objective function. This typically involves minimizing a loss function.

A convolutional neural network layer may apply a specified convolution operation. In the present example, three-dimensional convolution may be applied by the Conv3D and ConvTranspose3D data classes of PyTorch (e.g., as in versions 1.x and 2.x). Other layers may also be defined within a neural network library. These include both layers with trainable parameters and layers without trainable parameters. Examples of other layers include normalisation layers, dropout layers, and activation functions. Common activation functions include the sigmoid function, the tanh function, and Rectified Linear Units (RELUs—including parameterised RELUs). Many other activation functions exist and may be applied. A softmax activation may be applied to convert a set of logits or scores into a set of probability values that sum to 1. An activation function may be selected based on testing and preference. Activation functions may be omitted in certain circumstances, and/or form part of the internal structure of a neural network layer. Other structural components of a neural network architecture include skip connections and residual connections.

Neural network layers and architectures as described herein may be configured to be trained using an approach called backpropagation. This approach may be structured within training methods of the machine learning programming tools and libraries. When training, a training set is supplied that consists of pairs of input and output data. In this case, training sets may comprise three-dimensional CT imaging data and labelled voxels of that data (e.g., in terms of lung segmentation and/or fibrosis binary or integer/multi-class labels). In certain examples, a plurality of neural network layers and/or architectures may be communicatively coupled to form a compute graph. In particular cases, the layers and architectures, and even the whole system may be trained as a whole (sometimes referred to as “end-to-end” training).

In the training set, the output data is often called a “ground truth” label as it represents what the output should be. In the present examples, at least some of the “ground truth” labels may be labels as previously manually annotated by radiologists. During backpropagation, the neural network layers that make up each neural network architecture are initialized (e.g., with randomized weights) and then used to make a prediction using a set of input data from the training set (e.g., a so-called “forward” pass). The prediction is compared with the corresponding “ground truth” output data from the training set and an error is computed. The error may form part of a loss function. In the present examples, a loss function may be based on a comparison between a model prediction and a training “ground truth” segmentation label (e.g., one-hot labels indicating either “lung” or “fibrosis” for data elements). In certain preferred examples, the loss function uses a so-called dice loss based on the Sorensen-Dice coefficient. The dice loss may be computed as twice the number of positive determinations in the union of both ground truth and predicted data elements (e.g. the “fibrosis” labels for the set of voxels) divided by the sum of the number of positive determinations for the ground truth data elements and the number of positive determinations for the predicted data elements. A continuous dice loss may be used in cases where fibrosis labels comprise continuous values within a probability map.

If gradient descent methods are used, the error is used to determine a gradient of the loss function with respect to the parameters of the layers and architectures, where the gradient is then used to back propagate an update to the parameter values through the neural network architecture. Typically, the update is propagated according to the derivative of the weights of the neural network layers. For example, a gradient of the loss function with respect to the weights of the neural network layers may be determined and used to determine an update to the weights that minimizes the loss function. In this case, optimization techniques such as gradient descent, stochastic gradient descent, ADAM etc. may be used to adjust the weights. The chain rule and auto-differentiation functions may be applied to efficiently compute the gradient of the loss function, e.g. starting from the output of the layers and/or architectures and working back through the neural network layers of each neural network architecture in turn. In the present example, an ADAM optimiser is preferred. Weights may be initialised randomly.

The example of FIG. 3A is based on a three-dimensional U-Net architecture. In this example, three-dimensional lung imaging data 302 is received as a tensor (i.e., multi-dimensional matrix) in the form of a three-dimensional matrix of resolution 64-by-64-by-64 with one channel of greyscale data representing intensity values of the CT scan. It should be noted that actual implementations may have a higher resolution and additional layers depending on the capabilities of the implementing computer architecture. There are then four layers—layers 0 to 3—where processing moves down through the layers while reducing in resolution (in three-dimensions) and then moves up the layers while increasing in resolution (in three-dimensions). The architecture resembles a U-shape and operates on three-dimensional data, hence it may be seen as a particular implementation of a three-dimensional U-Net.

In FIG. 3A, the number of channels is shown in curly brackets (“{ . . . }”). These may be seen to be a set of “features” of each voxel at each resolution. Each data channel has a value at each three-dimensional location. Data is sequentially processed by seven data processing blocks 304 to 318. Each data processing block applies at least a three dimensional convolution neural network operation. The three dimensional convolution neural network operation has a set of trainable filter parameters. These parameters are learnt during neural network training, and the architecture (e.g., as shown in FIG. 3A) is trained as an end-to-end system.

Processing begins with left layer 0 processing block 304. This block 304 receives the 3D imaging data 302 and applies a three-dimensional convolution neural network operation. The 3D imaging data 302 is received as a tensor of size [1, 64, 64, 64] and is output as a (transformed) tensor of size [16, 32, 32, 32]—i.e. as a set of voxels at a lower resolution each having a feature vector of size 16. The output of the left layer 0 processing block 304 is provided to a left layer 1 processing block 306 and also passed across to the right side of the architecture via connection 332. The left layer 1 processing block 306 receives input data of size [16, 32, 32, 32], applies a three-dimensional convolution neural network operation, and outputs a tensor of size [32, 16, 16, 16]—i.e. as a set of voxels at a lower resolution each having a feature vector of size 32. The output of the left layer 1 processing block 306 is provided to a left layer 2 processing block 308 and also passed across to the right side of the architecture via connection 334. The left layer 2 processing block 308 receives input data of size [32, 16, 16, 16], applies a three-dimensional convolution neural network operation, and outputs a tensor of size [64, 8, 8, 8]—i.e. as a set of voxels at a lower resolution each having a feature vector of size 64.

At the bottom of the architecture is a lower portion 310 comprising a layer 3 processing block 312. The output of the left layer 2 processing block 308 is also passed across to the right side of the architecture via connection 336. The layer 3 processing block 312 receives input data of size [64, 8, 8, 8], applies a three-dimensional convolution neural network operation, and outputs a tensor of size [128, 8, 8, 8]—i.e. as a set of voxels at the same resolution each having a feature vector of size 128. At the output of the lower portion 310, the output of the layer 3 processing block 312 is concatenated with the data passed via connection 336 so as to provide a tensor of size [192, 8, 8, 8]. This tensor is input to a right layer 2 processing block 314, which applies a three-dimensional convolution neural network operation, and outputs a tensor of size [32, 16, 16, 16]—i.e. as a set of voxels at a higher resolution each having a feature vector of size 32. The tensor output from the right layer 2 processing block 314 is then concatenated with the data received along connection 334 so as to provide a tensor of size [64, 16, 16, 16]. This tensor is input to a right layer 1 processing block 316, which applies a three-dimensional convolution neural network operation, and outputs a tensor of size [16, 32, 32, 32]—i.e. as a set of voxels at a higher resolution each having a feature vector of size 16. The tensor output from the right layer 1 processing block 316 is then concatenated with the data received along connection 332 so as to provide a tensor of size [32, 32, 32, 32]. This tensor is then input to a right layer 0 processing block 318, which applies a three-dimensional convolution neural network operation, and outputs a tensor of size [2, 64, 64, 64]—i.e. as a set of voxels at a higher resolution each having a feature vector of size 2. This is then provided as the three-dimensional fibrosis segmentation data 320. In this case, a first dimension of the feature vector indicates a probability that a voxel forms part of a background and a second dimension of the feature vector indicates a probability that a voxel exhibits fibrosis. These two probability values for each voxel are then converted into a single segmentation map.

In certain examples, the architecture shown in FIG. 3A may also be used for the lung segmentation model (e.g., 120 in FIG. 1). In this case, the three-dimensional segmentation data 320 may comprise one or multiple channels indicating “lung” and/or portions of particular lung anatomy.

FIG. 3B shows an example 350 of components that may be used to implement one or more of the processing blocks 304 to 318. In certain cases, all processing blocks may have a common (i.e., shared) configuration. In other cases, the configuration may be similar but adapted at each level based on experimentation.

In FIG. 3B, block 360 represents the processing block in question. An input tensor is received and processed sequentially by two stages 362 and 364. A first stage 362 applies a three-dimensional convolution operation 372 (such as Conv3D or ConvTranspose3D) and a so-called ADN operation 374, where AND stands for Activation, Normalisation, and Dropout. In preferred cases, the ADN operation 374 comprises a three-dimensional normalisation, dropout, and a parameterised ReLU activation in series. Normalisation may use the InstanceNorm3d function in PyTorch. The output of the first stage 362 is passed to the second stage 364. The second stage 364 comprises a residual unit. The residual unit has similar components to the first stage 362 but with a residual connection 382. In the residual unit there are two parallel data flows—one along residual connection 382 and one along a lower path that comprises a three-dimensional convolution operation 384 (such as Conv3D or ConvTranspose3D) and a ADN operation 386. The second stage 364 is thus similar to a residual processing block as introduced in the ResNet architecture (see “Deep Residual Learning for Image Recognition” by He et al.—ArXiv 2015). The output from the second stage 364 is then provided as the output of the processing block 360.

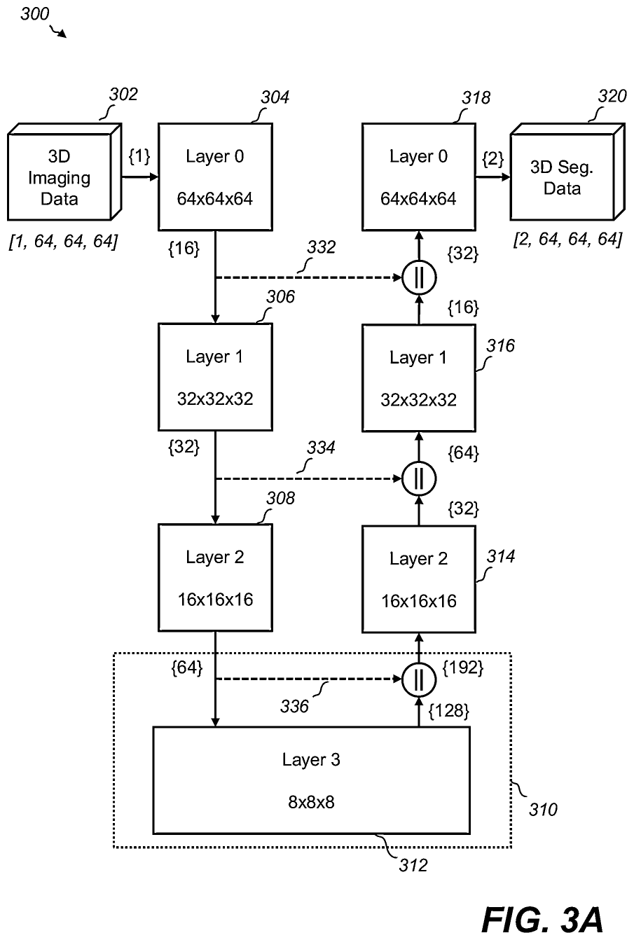

FIG. 4 shows an example method 400 of computing a lung fibrosis metric for a patient. This method may be performed using the system 102 of FIG. 1 and/or the model of FIG. 3A and/or using an alternative architecture.

At block 402, the method 400 comprises receiving initial lung imaging data for the patient. As described above, this may comprise obtaining two and/or three dimensional CT scan data, for example in the form of a NIfTI or DICOM file. The receiving may comprise, amongst others, accessing from a file system, accessing from memory, and/or receiving over a network connection. At block 404, the method comprises applying a trained neural network lung segmentation model to the initial lung imaging data to generate lung segmentation data, the lung segmentation data indicating which portions of the lung imaging data relate to lung features of the patient. This may comprise initiating a forward or “inference” pass of the trained neural network lung segmentation model using the initial lung imaging data as input data to the model. Lung segmentation data may comprise two or three-dimensional data with predicted annotations identifying data elements that correspond to general and/or specifically named lung areas. The lung segmentation data may be supplied at the same spatial resolution as the initial lung imaging data; in this case, the application of the trained neural network lung segmentation model may be seen as creating a label for each data element (e.g. pixel or voxel) of the initial lung imaging data. The lung segmentation may be provided as binary, multi-class (e.g., integer), probability, or logit data.

At block 406, the method 400 comprises applying the lung segmentation data to the initial lung imaging data to produce modified lung imaging data. This may comprise multiplying together (e.g., elementwise) data values for data elements of the lung segmentation data and data values for data elements of the initial lung imaging data. In the case that the lung segmentation data is provided as a probability map, this may comprise weighting the initial lung imaging data by a weight between 0 and 1 (or a weight computed as a function of the probability values).

At block 408, the method 400 comprises applying a trained neural network lung fibrosis model to the modified lung imaging data to generate fibrosis segmentation data. As for block 404, this may comprise initiating a forward or “inference” pass of the trained lung neural network fibrosis model using the modified lung imaging data as input data to the model. Block 408 may comprise processing the data using a similar pipeline to that shown in FIG. 3A (and/or processing blocks as per FIG. 3B). The fibrosis segmentation data indicates which portions of the modified lung imaging data relate to fibrosis features, e.g. in the form of binary, logit, or probability values.

At block 410, the method 400 comprises processing the fibrosis segmentation data in combination with the lung segmentation data to generate labelled voxel data. This may comprise multiplying (e.g., in an elementwise multiplication) the fibrosis segmentation data with the lung segmentation data. In other cases, it may comprise applying threshold and/or sampling operations to label voxels. The voxels may comprise voxels that correspond to voxels of the original lung imaging data. The labelled voxel data may comprise binary fibrosis and lung segmentation labels, i.e. values indicating which voxels are classified as relating to the lung and which as exhibiting fibrosis.

At block 412, the method comprises computing a fibrosis volume metric for the patient using the labelled voxel data the fibrosis volume metric representing a proportion of lung volume that exhibits fibrosis. This may comprise computing a ratio of the fibrosis-labelled voxels to the lung-labelled voxels. The fibrosis volume metric may be provided as a fraction (e.g., between 0 and 1) and/or as a percentage. Lung anatomy labels in the lung segmentation data may be used to determine fibrosis volume metrics for different anatomic portions of the lungs.

Lastly at block 414, the method 400 comprises providing the fibrosis volume metric as the lung fibrosis metric for the patient. This may comprise, amongst others, saving one or more values to a file, saving one or more values to memory, outputting one of more values via a graphical user interface, and/or transmitting one or more values to a remote network location.

In certain variations, the initial lung imaging data comprises three-dimensional computed tomography (CT) image data. In this case, or other cases, one or more of the lung segmentation data and the fibrosis segmentation data each comprise a set of feature vectors for portions of a three-dimensional volume containing a representation of the lungs of the patient. For example, these feature vectors may comprise one or more channels. The feature vectors for the lung segmentation data may comprise a single probability channel that indicates a “lung” classification and/or a series of probability channels that correspond to specific anatomic portions of the lung (such as one or more of lung lobes, segments, and lobules). The feature vectors for the fibrosis segmentation data may comprise a probability channel that indicates whether a voxel exhibits fibrosis features. The lung segmentation model and the lung fibrosis model may each comprise a three-dimensional convolutional neural network architecture. For example, this may be based on the architecture shown in FIGS. 3A and/or 3B. The two models may share the same three-dimensional convolutional neural network architecture. The lung segmentation model and the lung fibrosis model have different trained parameters. Example models having around 1 million trainable parameters and an architecture similar to that shown in FIGS. 3A and 3B performed well in testing, accurately mimicking an expert classification (as illustrated by the test output of the system that is shown in FIGS. 2A and 2B).

In certain variations, the three-dimensional convolutional neural network architecture is the same for both the lung segmentation model and the lung fibrosis model. In this case, the three-dimensional convolutional neural network architecture may comprise a three-dimensional UNet architecture (e.g., as shown in FIG. 3A) with a plurality of modules arranged to process three-dimensional data at different three-dimensional resolutions. In certain cases, voxels at each three-dimensional resolution are mapped to a defined volume. For example, each voxel may be deemed to relate to a particular volume in cubed millimetres, where different resolutions are mapped to different external volumes, representing volumes of scanned tissue. In this case, each processing module may comprise convolutional and residual units, e.g. as shown in FIG. 3B. Each of the convolutional and residual units may comprise a normalisation unit, a dropout unit, and an activation function unit.

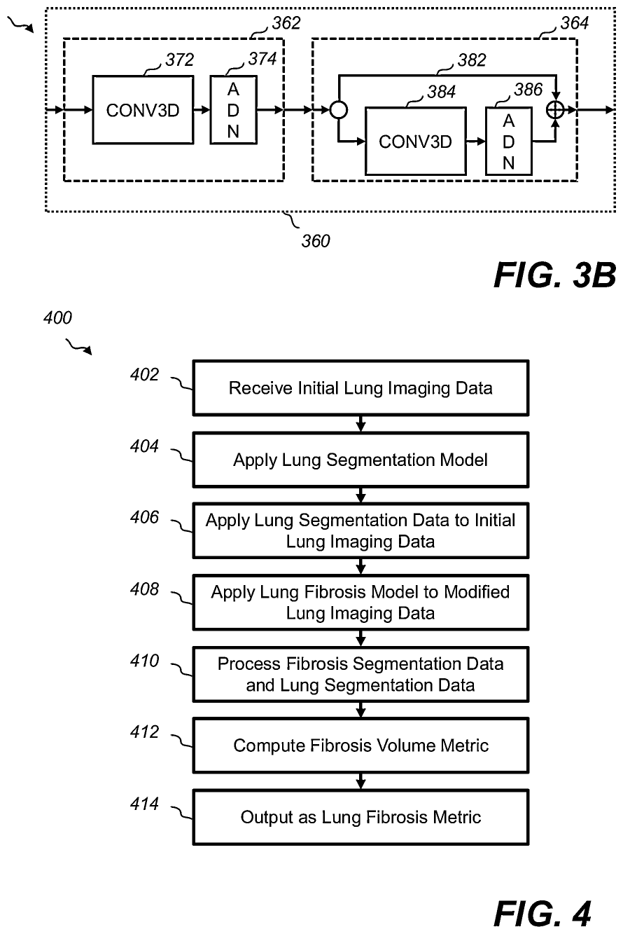

FIG. 5 shows an example method 500 of configuring a lung fibrosis metric system. The method of FIG. 5 may be used to train the neural network models shown in the system 102 of FIG. 1 and/or neural network models as configured according to the architecture of FIG. 3A.

At block 502, the method 500 comprises obtaining lung segmentation training data. The lung training data comprises lung imaging data with lung segmentation annotations. For example, this may comprise images representing CT scans with an extra pixel channel indicating pixels that relate to lung tissue and/or an integer pixel channel indicating a particular lobe (or other specific area of anatomy). It may also comprise a three-dimensional model with voxel annotations, such as provided by the NIfTI file format. The lung training data may comprise a directory of files or a database of tensor data. The lung segmentation annotations may be derived from manual “expert” annotations, such as digitalised hand segmentations and/or segmentations performed by a radiologist on two or three-dimensional displayed data.

At block 504, the method 500 comprises training a neural network lung segmentation model based on the lung segmentation training data to produce a set of trained lung segmentation model parameters. Many machine learning programming tools and libraries (such as PyTorch and TensorFlow) have built in methods for training that may be applied to a defined training dataset and a defined model. The training comprising optimising a loss function, the loss function being computed based on a comparison of a ground-truth lung segmentation annotation and a predicted lung segmentation annotation from the lung segmentation neural network model. Different loss functions may be configured. In a preferred example, a dice loss function (as described above) is used. This may be defined as a custom loss function or selected from a pre-defined set of available loss functions.

At block 506, the method 500 comprises obtaining fibrosis training data. This is training data for the fibrosis model, such as the fibrosis model 140 in FIG. 1 and/or the architecture shown in FIG. 3A. The fibrosis training data comprising lung imaging data with fibrosis segmentation annotations. The lung imaging data may be in the same format as the lung imaging data obtained at block 502 or may comprise a different set of data. The fibrosis training data may comprise images representing CT scans with an extra pixel channel indicating pixels that relate to fibrosis within the lungs. It may also comprise a three-dimensional model with voxel annotations, such as provided by the NIfTI file format. The fibrosis annotation may comprise a single one-hot value for each voxel within the three-dimensional model. The fibrosis training data may comprise a directory of files or a database of tensor data.

At block 508, the method 500 comprises training a neural network lung fibrosis model based on the fibrosis training data. The neural network lung fibrosis model may comprise the fibrosis model 140 as described with reference to FIG. 1, and/or it may be arranged based on the example of FIGS. 3A and 3B. Again, training may be based on pre-defined methods within machine learning programming tools and libraries. The training comprising optimising a loss function, the loss function being computed based on a comparison of a ground-truth fibrosis segmentation annotation and a predicted fibrosis annotation from the neural network lung fibrosis model. The loss function may again comprise a dice loss function (as described above). In one case, the neural network lung fibrosis model may output a set of probability values, where each pixel or voxel in a training sample has an associated probability value. These probability values indicate a likelihood of fibrosis. These may be compared to a set of ground truth one-hot fibrosis annotations that are defined in the training data for each data element. In this case, the dice loss may comprise a continuous dice loss that is based on a comparison between the set of probability values and the set of one-hot values. In other cases, other segmentation definition formats may be used and a different loss function selected accordingly. The dice loss function was found to work well in testing.

Within block 508, the predicted fibrosis annotation is computed via a forward or inference pass of the neural network lung fibrosis model. This may be performed as described with reference to one or more of FIGS. 1, 3A, 3B, and 4. For example, the following operations may be performed for each data sample in the fibrosis training data. First, lung segmentation data may be computed using the neural network lung segmentation model that is instantiated with the trained lung segmentation model parameters and applied to a current data sample. Next, the data sample from the lung imaging data is pre-processed using the lung segmentation data to generate a modified sample. Then, the neural network lung fibrosis model is applied in a training mode to the modified sample to generate the predicted fibrosis annotation. Using a pre-trained or fixed set of lung segmentation model parameters may aid the stability of the training procedure, finding a suitable local minima more quickly than joint training. Furthermore, it may be easier to obtain independent lung segmentation and fibrosis datasets. However, in other examples, joint training may alternatively be performed. Joint training may train the complete system of FIG. 1 in an end-to-end manner (e.g., as opposed to just the complete system of FIG. 3A). For joint training a combined training set is needed where data elements are provided with both lung and fibrosis labels.

In certain cases, a focal loss may be applied as well as, or instead of, the dice loss. A focal loss function is based on a cross-entropy loss where a weighting or “focussing” parameter is defined as a model hyperparameter to give more prominence to certain class classifications. In the present examples, the focal loss may be applied to give more prominence to positive “fibrosis” data element classifications. This may help where there are fewer positive “fibrosis” labels within a training sample (e.g., even for a patient exhibiting signs of fibrosis, there may be a low percentage of data elements deemed to relate to fibrotic features—as can be seen in FIGS. 2A and 2B). A focal loss may also be applied to a multi-class label for finer anatomic labelling of lung portions when training the lung segmentation model. There may be a weighting parameter for each class. This may help if certain anatomic areas are larger than others leading to a class imbalance across a two or three dimensional data sample. Certain test examples were trained with a dice loss and certain test examples were trained with a combination of dice and focal losses. Both provided good training performance. The exact loss function, based on these teaching, may be selected based on the training data and neural network architecture.

As described above, in certain examples, training may consist of presenting the lung fibrosis model with new CT images, and the model is configured to quantify these new images according to fibrosis embedded from prior training. In certain cases, additional “human-in-the-loop” training feedback may be provided. In these cases, the output from the lung fibrosis model may be presented to an expert radiology team. Any error in the output image classification may be corrected by the team or via other semiautomated means, and the correction may be fed back into the training procedure as part of the loss function. In certain cases, particular hand-corrected errors may be over weighted within the loss function to enhance their impact on optimization of the model parameters. In these cases, the model may be updated in an iterative manner, where an update repeats until the lung fibrosis model reaches a certain loss value based on expert intervention and the choice of challenging images.

Examples presented herein use a supervised training approach. However, training samples may comprise, or be enhanced with, synthetic training examples to facilitate training. Synthetic training examples may be based on automated or semi-automated methods. For example, if CT imaging data is available in the form of a three-dimensional annotated model and the neural network models are configured to receive two-dimensional inputs, then additional two dimensional slices may be generated from the three-dimensional model in additional to available two-dimensional images with annotations. Similarly, in the case that the neural network models are configured to receive three-dimensional inputs and only sets of two-dimensional annotated images are available, machine learning approaches (e.g., based on three-dimensional interpolation or sample selection) may be used to generate three-dimensional annotated data samples from the original two-dimensional annotated images. In certain cases, if physical images with manual hand annotations are available these may be digitalised to generate digital copies for use in training.

The convolutional neural network architectures described herein may use kernels and/or filters to extract features in two or (preferably) three dimensions. Those skilled in the art will understand that variations may be applied to layer configurations depending on the data being processed. The various stages of the convolutional neural network may employ different configurations. The examples provided in this detailed disclosure were found to result in a reliable and accurate lung fibrosis metric; however, adaptations to these examples may be made to accommodate future improvements in training and/or inference will remaining consistent with the general concepts described herein. Suitable hyperparameters of the neural networks models may be set based on library defaults and/or based on systematic experimentation. Hyperparameters include, but are not limited to, learning rate, batch size and a number of training epochs. These may be optimized using searches and visualizations. Reference to a “trained” neural network model herein is deemed to relate to a neural network model that has undertaken training to optimise a set of parameters associated with the model (e.g., by way of the optimisation of a loss function as discussed herein). Model parameters may be supplied as configuration data to instantiate particular models.

Convolutional neural networks are described herein may learn to segment fibrosis based on morphological and radiological appearances such as ground glass, reticulation and honeycombing. Preferred three-dimensional neural network models may learn patterns that extend in two and three dimensions.

In certain variations, a multi-class fibrosis model may be provided wherein an output fibrosis segmentation indicates probabilities for one or more of a plurality of different types of fibrosis. These different type of fibrosis may include typical interstitial pneumonia, non-specific interstitial pneumonia and indeterminate fibrosis. A multi-class fibrosis neural network model may be training in a similar manner to the examples discussed herein, but with a cross-entropy or focal loss function. Being able to determine different fibrosis types may be beneficial as each type may have a different response to treatment and prognosis.

In the context of using the fibrosis model in relation to clinical application, trial, testing, or screening described herein, it would be understood that the application, trial, testing, or screening are under controlled conditions and with the informed consent of the patient in the event that the trial, testing, or screen will be performed on the patient.

In certain examples described herein, a system is presented that comprises a combination of two machine learning models: 1) a lung extractor model (i.e., a lung lobe model) and 2) a fibrosis model. In certain cases, the lung extractor model may be based on the lung lobe model described in WO2023/281252 A2. In other cases, the lung extractor model may be based on the examples described herein. Both models may be arranged as convolutional neural network architectures, with each comprising at least one network layer, filters, and/or kernels. In examples, starting from a full-size CT scan or image, the lung extractor model may be applied first to generate a segmentation mask of the lungs. This mask may then be used as additional input by the lung fibrosis model in order to focus on the patterns inside the lungs. The fibrosis model then generates segmentations representing fibrosis patterns.

In certain variations, the fibrosis segmentation may be used as a mask to extract features. Features may then be clustered using a clustering algorithm, e.g. to detected continuous or related portions of fibrosis. In one case, a form of connected component analysis may additionally and/or alternatively be applied. Where clustering is applied, features derived from the fibrosis segmentation may be clustered using a clustering algorithm such as a k-means algorithm with a fixed number of clusters. The identified clusters (e.g., in the form of unique cluster identifiers) may then be used as segmentation labels to generate segmentation maps of subpatterns found within the fibrosis segmentation mask.

In certain variations, the systems, methods, and/or models may be applied in a clinical context. For example, the generated lung fibrosis metric may be used to support or facilitate clinical trials. In this case, a system may be implemented with data storage and at least one processor. The processor may be configured to receive CT scans of a patient with lung disease. The CT scans may be collected at one time point. At least one time point is collected before the patient has not been administered a drug. One or more time points are then collected after the patient has been administered the drug. The system in this case generates a data set associated with the progression of the lung disease from the CT scans by generating a lung fibrosis metric for each time point. This data set may be analysed to determine a drug-induced effect. The determination may support a clinical study for a particular therapy of a drug candidate. In one case, CT scans are collected from at least two time points, at least one time point collected before the patient has not been administered a drug, and one or more time points after the patient has been administered the drug. In this case, an automatic and quantitative assessment of fibrosis of the lungs is performed by the system (e.g., the system 104 of FIG. 1). The lung fibrosis metric is then used to analyse and quantify the response to treatment, e.g. may be used to determine if the drug is slowing down the fibrosing process compared to placebo or standard of care treatment. Fibrosis metrics may be computed as described herein for different lung portions (e.g., lobes) and progress further tracked at the lower level of these portions.

In certain variations, a method for measuring the extent and distribution of lung fibrosis in clinical practice may use the systems, methods, and/or models described herein to monitor a patient more accurately than the subjective and semiquantitative assessment performed by a radiologist. The present examples may be used to help identify and/or track a lung disease such as Idiopathic Pulmonary Fibrosis (IPF) or a related interstitial lung disease (ILD). In one case, the generated lung fibrosis metric may be used for improving clinical trial enrichment, wherein said at least one result is applied to select a patient with the lung disease at a desired disease progression.

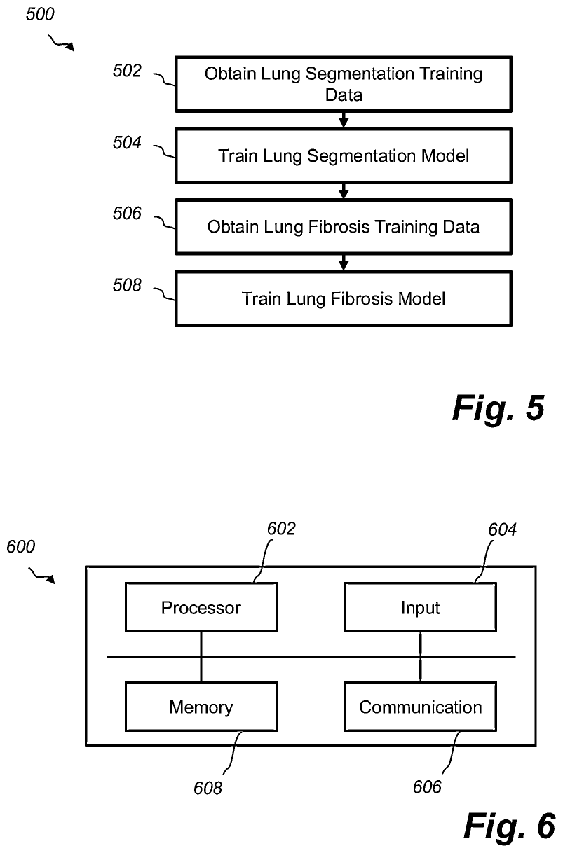

FIG. 6 is a block diagram illustrating an example computing apparatus/system 600 that may be used to implement one or more aspects of the system(s), apparatus, method(s), and/or process(es) combinations thereof, modifications thereof, and/or as described with reference to FIGS. 1 to 5 and/or as described herein. Computing apparatus/system 600 includes one or more processor unit(s) 602, an input/output unit 604, communications unit/interface 606, a memory unit 608 in which the one or more processor unit(s) 602 are connected to the input/output unit 604, communications unit/interface 606, and the memory unit 608. In some examples, the computing apparatus/system 600 may be a server, or one or more servers networked together. In some examples, the computing apparatus/system 600 may be a computer or supercomputer/processing facility or hardware/software suitable for processing or performing the one or more aspects of the system(s), apparatus, method(s), and/or process(es) combinations thereof, modifications thereof, and/or as described with reference to FIGS. 1 to 5 and/or as described herein. The communications interface 606 may connect the computing apparatus/system 600, via a communication network, with one or more services, devices, server system(s), cloud-based platforms, and/or systems for implementing subject-matter databases and/or knowledge graphs. The memory unit 608 may store one or more program instructions, code or components such as, by way of example only but not limited to, an operating system and/or code/component(s) associated with the process(es)/method(s) as described with reference to FIGS. 1 to 5, additional data, applications, application firmware/software and/or further program instructions, code and/or components associated with implementing the functionality and/or one or more function(s) or functionality associated with one or more of the method(s) and/or process(es) of the device, service and/or server(s) hosting the process(es)/method(s)/system(s), apparatus, mechanisms and/or system(s)/platforms/architectures for implementing the invention as described herein, combinations thereof, modifications thereof, and/or as described with reference to at least one of the Figure(s) 1 to 5.

In the examples, and aspects of the invention as described above such as process(es), method(s), system(s) and/or apparatus may be implemented on and/or comprise one or more cloud platforms, one or more server(s) or computing system(s) or device(s). A server may comprise a single server or network of servers; the cloud platform may include a plurality of servers or network of servers. In some examples the functionality of the server and/or cloud platform may be provided by a network of servers distributed across a geographical area, such as a worldwide distributed network of servers, and a user may be connected to an appropriate one of the network of servers based upon a user location and the like.

The described embodiments of the invention a system, process(es), method(s) and/or apparatus according to the invention and/or as herein described may be implemented as any form of a computing and/or electronic device. Such a device may comprise one or more processors which may be microprocessors, graphical processing units, controllers or any other suitable type of processors for processing computer executable instructions to control the operation of the device in order to gather and record routing information. Reference to “processors” in relation to FIG. 1 may refer to functional modules that are implemented using one or more processors executing computer program code that defines the functional modules. Such “processors” may thus be implemented using one or more processors but need not be provided as separate hardware processors. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

The present neural network models may be implemented using one or more Graphical Processing Units (GPU) to speed up training and/or inference (e.g., using lower level functionality based on the Compute Unified Device Architecture—CUDA).

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, IoT devices, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one example or may relate to several examples. The examples are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The Figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of preferred examples are provided to indicate to the skilled person various aspects of the present invention and that various modifications may be made by those skilled in the art.

All publications set out herein are deemed to be incorporated by reference.

What has been described above includes examples. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A system for computing a lung fibrosis metric for a patient, the system comprising:
an input interface to receive initial lung imaging data for the patient; and
a trained neural network lung segmentation model to generate lung segmentation data from the initial lung imaging data, the lung segmentation data indicating which portions of the lung imaging data relate to lung features of the patient;
a fibrosis model pre-processor to apply the lung segmentation data to the lung imaging data to produce modified lung imaging data;
a trained neural network lung fibrosis model to generate fibrosis segmentation data from the modified lung imaging data, the fibrosis segmentation data indicating which portions of the modified lung imaging data relate to fibrosis features;
a fibrosis model post-processor to process the fibrosis segmentation data in combination with the lung segmentation data to generate labelled voxel data;
a fibrosis metric processor to use the labelled voxel data to compute a fibrosis volume metric for the patient, the fibrosis volume metric representing a proportion of lung volume that exhibits fibrosis; and
an output interface to provide the fibrosis volume metric as the lung fibrosis metric for the patient,
wherein the lung segmentation data and the fibrosis segmentation data each comprise a set of feature vectors for portions of a three-dimensional volume.

2. The system of claim 1, wherein the initial lung imaging data comprises one or more of:
a set of computed tomography (CT) images; and
three-dimensional CT image data.

3. The system of claim 1, wherein the fibrosis model pre-processor is configured to perform a matrix multiplication using the lung segmentation data and the lung imaging data to produce the modified lung imaging data.

4. The system of claim 1, wherein the lung segmentation model is configured to generate lung segmentation data with a set of channels that respectively indicate different lung lobe portions, and wherein the fibrosis metric processor is configured to compute one or more fibrosis volume metrics for one or more different lobe portions of the patient.

5. The system of claim 1, wherein the lung segmentation model and the lung fibrosis model each comprise a three-dimensional convolutional neural network architecture, the lung segmentation model and the lung fibrosis model having different trained parameters.

6. The system of claim 5, wherein the three-dimensional convolutional neural network architecture is the same for both the lung segmentation model and the lung fibrosis model, the three-dimensional convolutional neural network architecture comprises a three-dimensional U-Net architecture with a plurality of modules arranged to process three-dimensional data at different three-dimensional resolutions, wherein voxels at each three-dimensional resolution are mapped to a defined volume, each module comprising convolutional and residual units.

7. The system of claim 6, wherein each of the convolutional and residual units comprise:
a normalisation unit;
a dropout unit; and
an activation function unit.

8. The system of claim 1, wherein the fibrosis segmentation data comprises a three-dimensional voxel matrix with binary fibrosis features.

9. The system of claim 1, wherein the lung segmentation model is trained based on a set of human-annotated lung imaging data indicating different lung portions and the lung fibrosis model is trained based on a set of human-annotated lung imaging data indicating portions of fibrosis.

10. The system of claim 1, wherein the lung fibrosis metric is computed over time to evaluate progression of Idiopathic Pulmonary Fibrosis (IPF).

11. A computer-implemented method of computing a lung fibrosis metric for a patient, the method comprising:
receiving initial lung imaging data for the patient; and
applying a trained neural network lung segmentation model to the initial lung imaging data to generate lung segmentation data, the lung segmentation data indicating which portions of the lung imaging data relate to lung features of the patient, the lung segmentation data comprising a set of feature vectors for portions of a three-dimensional volume;
applying the lung segmentation data to the lung imaging data to produce modified lung imaging data;
applying a trained neural network lung fibrosis model to the modified lung imaging data to generate fibrosis segmentation data, the fibrosis segmentation data indicating which portions of the modified lung imaging data relate to fibrosis features, the fibrosis segmentation data comprising a set of feature vectors for portions of a three-dimensional volume;
processing the fibrosis segmentation data in combination with the lung segmentation data to generate labelled voxel data;
computing a fibrosis volume metric for the patient using the labelled voxel data, the fibrosis volume metric representing a proportion of lung volume that exhibits fibrosis; and
providing the fibrosis volume metric as the lung fibrosis metric for the patient.

12. The computer-implemented method of claim 11, wherein:
the initial lung imaging data comprises three-dimensional computed tomography (CT) image data;
applying the lung segmentation data to the lung imaging data to produce modified lung imaging data comprises applying a matrix multiplication to the lung segmentation data and the lung imaging data; and
the lung segmentation model and the lung fibrosis model each comprise a three-dimensional convolutional neural network architecture, the lung segmentation model and the lung fibrosis model having different trained parameters.

13. The computer-implemented method of claim 12, wherein the three-dimensional convolutional neural network architecture is the same for both the lung segmentation model and the lung fibrosis model, the three-dimensional convolutional neural network architecture comprises a three-dimensional U-Net architecture with a plurality of modules arranged to process three-dimensional data at different three-dimensional resolutions, wherein voxels at each three-dimensional resolution are mapped to a defined volume, each module comprising convolutional and residual units, wherein each of the convolutional and residual units comprise a normalisation unit, a dropout unit, and an activation function unit.

14. A computer-implemented method of configuring a lung fibrosis metric system, the method comprising:
obtaining lung segmentation training data comprising lung imaging data with lung segmentation annotations, the lung segmentation data comprising a set of feature vectors for portions of a three-dimensional volume; and
training a neural network lung segmentation model based on the lung segmentation training data to produce a set of trained lung segmentation model parameters, said training comprising optimising a loss function, the loss function being computed based on a comparison of a ground-truth lung segmentation annotation and a predicted lung segmentation annotation from the neural network lung segmentation model;
obtaining fibrosis training data comprising lung imaging data with fibrosis segmentation annotations; and
training a neural network lung fibrosis model based on the fibrosis training data, said training comprising optimising a loss function, the loss function being computed based on a comparison of a ground-truth fibrosis segmentation annotation and a predicted fibrosis annotation from the neural network lung fibrosis model, the predicted fibrosis annotation being computed by:
computing lung segmentation data using the lung segmentation model instantiated with the trained lung segmentation model parameters as applied to a sample from the lung imaging data;
pre-processing the sample from the lung imaging data using the lung segmentation data to generate a modified sample; and
applying the lung fibrosis model in a training mode to the modified sample to generate the predicted fibrosis annotation, the predicted fibrosis annotation being generated from set of feature vectors for portions of a three-dimensional volume that are output by the lung fibrosis model in the training mode.

15. The computer-implemented method of claim 14, wherein pre-processing the sample from the lung imaging data using the lung segmentation data to generate a modified sample comprises performing a matrix multiplication using the lung segmentation data and the lung imaging data.
